(19) United States
(12) Patent Application Publication
(10) Pub. No.: US 2026/0028296 A1
(43) Pub. Date: Jan. 29, 2026

(54) REACTOR SYSTEM FOR ACETYLENE ABSORPTION AND SELECTIVE HYDROGENATION

(71) Applicant: Chevron U.S.A. Inc., San Ramon, CA (US)

(72) Inventors: Huping Luo, Moraga, CA (US); Xiaoying Ouyang, Albany, CA (US); Christopher Declan Lane, El Cerrito, CA (US); Howard Steven Lacheen, Richmond, CA (US)

(21) Appl. No.: 19/214,477

(22) Filed: May 21, 2025

Related U.S. Application Data
(60) Provisional application No. 63/674,414, filed on Jul. 23, 2024.

(51) Int. Cl. C07C 5/09; B01J 38/48; C07C 7/10

(52) U.S. Cl. CPC C07C 5/09; B01J 38/48; C07C 7/10

(57) ABSTRACT

A system including a slurry reactor configured to receive an acetylene-rich gas stream and a hydrogen stream as one stream or as separate streams flowing upwards and a slurry catalyst comprising catalyst particles and a liquid solvent flowing downwards, wherein the liquid solvent extracts acetylene in the acetylene-rich gas stream and hydrogen in the hydrogen stream and at least a portion of the acetylene is converted to ethylene in the presence of the slurry catalyst and the hydrogen under hydrogenation reaction conditions to generate an acetylene-lean gas effluent and a spent slurry catalyst including spent catalyst particles and the liquid solvent.

### PRIORITY CLAIM

The present application claims priority to U.S. Provisional Patent Application Ser. No. 63/674,414, entitled “Reactor System for Acetylene Absorption and Selective Hydrogenation,” filed Jul. 23, 2024, the content of which is incorporated by reference herein in its entirety.

### BACKGROUND

The ongoing search for alternatives to crude is increasingly driven by a number of factors. These include diminishing petroleum reserves, higher anticipated energy demands, and heightened concerns over greenhouse gas emissions from sources of non-renewable carbon. In view of its abundance in natural gas reserves, as well as in gas streams obtained from biological sources (biogas), natural gas has become the focus of a number of possible routes for providing liquid hydrocarbons. Natural gas occurs underground and is present as a gas when it comes out of the ground. Natural gas primarily consists of methane (CH₄), and additionally some other compounds such as ethane (C₂H₆) and propane (C₃H₈). Accordingly, converting light hydrocarbons such as methane to high value products such as hydrogen, olefins and aromatics has become an attractive option.

### SUMMARY

In accordance with an illustrative embodiment, a system comprises:
a slurry reactor configured to receive an acetylene-rich gas stream and a hydrogen stream as one stream or as separate streams flowing upwards to a reaction zone and a slurry catalyst comprising catalyst particles and a liquid solvent flowing downwards to the reaction zone, wherein the liquid solvent extracts acetylene in the acetylene-rich gas stream and hydrogen in the hydrogen stream and at least a portion of the acetylene is converted to ethylene in the presence of the slurry catalyst and the hydrogen under hydrogenation reaction conditions to generate an acetylene-lean gas effluent and a spent slurry catalyst comprising spent catalyst particles and the liquid solvent.

In accordance with another illustrative embodiment, a continuous process comprises:
passing an acetylene-rich gas stream and a hydrogen stream as one stream or as separate streams to a slurry reactor flowing upwards to a reaction zone in the slurry reactor,
passing a slurry catalyst comprising catalyst particles and a liquid solvent to the slurry reactor flowing downwards to the reaction zone in the slurry reactor, and
processing acetylene in the acetylene-rich gas stream and hydrogen in the hydrogen stream to convert at least a portion of the acetylene to ethylene in the presence of the slurry catalyst and the hydrogen under hydrogenation reaction conditions to generate an acetylene-lean gas effluent and a spent slurry catalyst comprising spent catalyst particles and the liquid solvent.

### BRIEF DESCRIPTION OF THE DRAWINGS

In combination with the accompanying drawings and with reference to the following detailed description, the features, advantages, and other aspects of the implementations of the present disclosure will become more apparent, and several implementations of the present disclosure are illustrated herein by way of example but not limitation. The principles illustrated in the example embodiments of the drawings can be applied to alternate processes and apparatus. Additionally, the elements and features shown in the drawings are not necessarily to scale, emphasis instead being placed upon clearly illustrating the principles of the example embodiments. Certain dimensions or positions may be exaggerated to help visually convey such principles. In the drawings, the same reference numerals used in different embodiments designate like or corresponding, but not necessarily identical, elements. In the accompanying drawings:

FIG. 1A illustrates a schematic diagram of a system and process for the selective absorption and hydrogenation of acetylene in an acetylene-rich gas stream to ethylene in an acetylene absorption and hydrogenation slurry reactor operating with a catalyst and solvent recycle loop, according to an illustrative embodiment.

FIG. 1B illustrates a schematic diagram of a system and process for the selective absorption and hydrogenation of acetylene in an acetylene-rich gas stream to ethylene in a series of acetylene absorption and hydrogenation slurry reactors operating with a catalyst and solvent recycle loop, according to an illustrative embodiment.

### DETAILED DESCRIPTION

Various illustrative embodiments described herein are directed to systems and processes for converting an acetylene-rich gas stream into a hydrogenation effluent comprising ethylene.

### Definitions

To define more clearly the terms used herein, the following definitions are provided. Unless otherwise indicated, the following definitions are applicable to this disclosure. If a term is used in this disclosure but is not specifically defined herein, the definition from the IUPAC Compendium of Chemical Terminology can be applied, as long as that definition does not conflict with any other disclosure or definition applied herein or render indefinite or non-enabled any claim to which that definition is applied. To the extent that any definition or usage provided by any document incorporated herein by reference conflicts with the definition or usage provided herein, the definition or usage provided herein controls.

While systems and processes are described in terms of “comprising” various components or steps, the systems and processes can also “consist essentially of” or “consist of” the various components or steps, unless stated otherwise.

The terms “a,” “an,” and “the” are intended to include plural alternatives, e.g., at least one. The terms “including,” “with,” and “having,” as used herein, are defined as comprising (i.e., open language), unless specified otherwise.

Various numerical ranges are disclosed herein. When Applicant discloses or claims a range of any type, Applicant's intent is to disclose or claim individually each possible number that such a range could reasonably encompass, including end points of the range as well as any sub-ranges and combinations of sub-ranges encompassed therein, unless otherwise specified. For example, all numerical end points of ranges disclosed herein are approximate, unless excluded by proviso.

Values or ranges may be expressed herein as “about,” from “about” one particular value, and/or to “about” another particular value. When such values or ranges are expressed, other embodiments disclosed include the specific value recited, from the one particular value, and/or to the other particular value. Similarly, when values are expressed as approximations, by use of the antecedent “about,” it will be understood that the particular value forms another embodiment. It will be further understood that there are a number of values disclosed therein, and that each value is also herein disclosed as “about” that particular value in addition to the value itself. In another aspect, use of the term “about” means±20% of the stated value, ±15% of the stated value, ±10% of the stated value, ±5% of the stated value, ±3% of the stated value, or ±1% of the stated value.

Applicant reserves the right to proviso out or exclude any individual members of any such group of values or ranges, including any sub-ranges or combinations of sub-ranges within the group, that can be claimed according to a range or in any similar manner, if for any reason Applicant chooses to claim less than the full measure of the disclosure, for example, to account for a reference that Applicant may be unaware of at the time of the filing of the application. Further, Applicant reserves the right to proviso out or exclude any members of a claimed group.

The term “continuous” as used herein shall be understood to mean a system that operates without interruption or cessation for a period of time, such as where reactant(s) and catalyst(s) are continually fed into a reaction zone and products are continually or regularly withdrawn without stopping the reaction in the reaction zone.

A “fresh catalyst” as used herein denotes a catalyst which has not previously been used in a catalytic process.

A “spent catalyst” as used herein denotes a catalyst that has less activity at the same reaction conditions (e.g., temperature, pressure, inlet flows) than the catalyst had when it was originally exposed to the process. This can be due to a number of reasons, several non-limiting examples of causes of catalyst deactivation are coking or carbonaceous material sorption or accumulation, steam or hydrothermal deactivation, metals (and ash) sorption or accumulation, attrition, morphological changes including changes in pore sizes, cation or anion substitution, and/or chemical or compositional changes.

A “regenerated catalyst” as used herein denotes a catalyst that had become spent, as defined above, and was then subjected to a process that increased its activity to a level greater than it had as a spent catalyst. This may involve, for example, reversing transformations or removing contaminants outlined above as possible causes of reduced activity. The regenerated catalyst typically has an activity that is equal to or less than the fresh catalyst activity.

The term “hydroconverting” or “hydroconversion,” as used herein refers to any process in which hydrocarbons are processed or treated in the presence of a hydrogen stream and a catalyst. Representative examples of hydroconverting include hydrocracking, hydrotreating, hydrogenation, deoxygenation, desulfurization, denitrogenation, demetallization, dechlorination, decarboxylation, decarbonylation, dearomatization or a combination thereof.

The term “zone” can refer to an area including one or more equipment items and/or one or more sub-zones. Equipment items can include one or more reactors or reactor vessels, absorption units, separation vessels, distillation towers, heaters, heat exchangers, pipes, pumps, compressors, and controllers. Additionally, an equipment item, such as a reactor, dryer, or vessel, can further include one or more zones or sub-zones.

The term “effluent” refers to a stream that is passed out of a reactor, a reaction zone, or a separation unit following a particular reaction or separation. Generally, an effluent has a different composition than the stream that entered the reactor, reaction zone, or absorption unit. It should be understood that when an effluent is passed to another component or system, only a portion of that effluent may be passed. For example, a slipstream may carry some of the effluent away, meaning that only a portion of the effluent may enter the downstream component or system.

The term “primarily” shall be understood to mean an amount greater than 50%, e.g., 50.01 to 100%, or any range between, e.g., 51% to 95%, 75% to 90%, at least 60%, at least 70%, at least 80%, etc.

For any figure shown and described herein, one or more of the components may be omitted, added, repeated, and/or substituted. Additionally, it should be understood that in certain cases components of the example systems can be combined or can be separated into subcomponents. Accordingly, embodiments shown in a particular figure should not be considered limited to the specific arrangements of components shown in such figure. Further, if a component of a figure is described but not expressly shown or labeled in that figure, the label used for a corresponding component in another figure can be inferred to that component. Conversely, if a component in a figure is labeled but not described, the description for such component can be substantially the same as the description for the corresponding component in another figure.

Direct conversion of light hydrocarbons such as methane (CH₄), ethane and propane under non-oxidative conditions can produce higher molecular weight hydrocarbons, such as olefins, alkynes and aromatics (e.g., benzene), as value-added chemicals and at the same time produce hydrogen that can be used to make, for example, fuel. Hydrogen is one of the more important options for future clean energy. However, the desired product selectivity obtained from the direct conversion processes will depend on the particular type of catalyst as well as reaction condition. In general, this reaction is highly endothermic with an enthalpy of about 90 KJ/mol of CH₄ or 60 KJ/mol of H₂, and the exact value of the reaction heat will depend on the desired product distribution. It is also an equilibrium limited reaction, and high temperatures are usually required to achieve a CH₄ conversion that would be practical for commercial applications. For example, to be commercially practical, maintaining a reactor at a temperature range of 600° C. to 1200° C. is required to achieve an acceptable methane conversion.

The required heat creates other practical challenges. For example, under such temperature conditions, the production of coke or solid carbon in the reactor becomes common, which can significantly reduce the yield of high value products, as well as cause significant operational issues such as plugging of the reactor and catalyst deactivation. Such high temperatures also can require expensive materials for the reactor and can make the design of the reactor challenging.

As a further example, under such high temperature conditions, a substantial amount of acetylene will also be produced during the conversion process. Acetylene and ethylene are hydrocarbons with the formula C₂H₂ and C₂H₄, respectively. They are widely used in the chemical industry, and their worldwide production exceeds that of any other organic compound. In the United States and Europe alone, approximately 90% of ethylene is used to produce ethylene oxide, ethylene dichloride, ethyl-benzene and polyethylene. However, although acetylene could be a high value final product, it is highly unstable. Therefore, separating and purifying acetylene to meet acetylene product specifications could significantly complicate the overall process.

In view of these challenges, there is a need for solutions to handle acetylene produced during the conversion process to reduce the associated safety risk as well as simplify the overall process. In addition, it would be advantageous for the reactor design for this process to have the capability to (1) provide the reaction heat needed to maintain an optimized temperature profile to achieve high conversion and (2) regenerate and recycle the catalyst being used. It would further be advantageous if such solutions are more energy efficient than existing approaches to produce hydrogen and value-added chemicals.

Illustrative embodiments address the above and other issues with existing providing reactor systems and processes for converting an acetylene-rich gas stream into a hydrogenation effluent comprising ethylene with a catalyst and solvent recycle loop. The systems and processes provide many advantages, examples of which are mentioned herein. For example, the non-limiting illustrative embodiments described herein overcome the drawbacks discussed above by providing slurry reactor systems and processes for converting a high concentration acetylene-rich gas stream into a hydrogenation effluent comprising ethylene at high conversion and high selectivity. Specifically, the non-limiting illustrative embodiments described herein are directed to systems and processes to selectively absorb acetylene from a mixed gas feedstock containing at least a high concentration of acetylene, ethylene, hydrogen, and other light hydrocarbon gases, followed by converting the absorbed acetylene into ethylene, to achieve high acetylene conversion and high ethylene selectivity with a catalyst and solvent recycle loop.

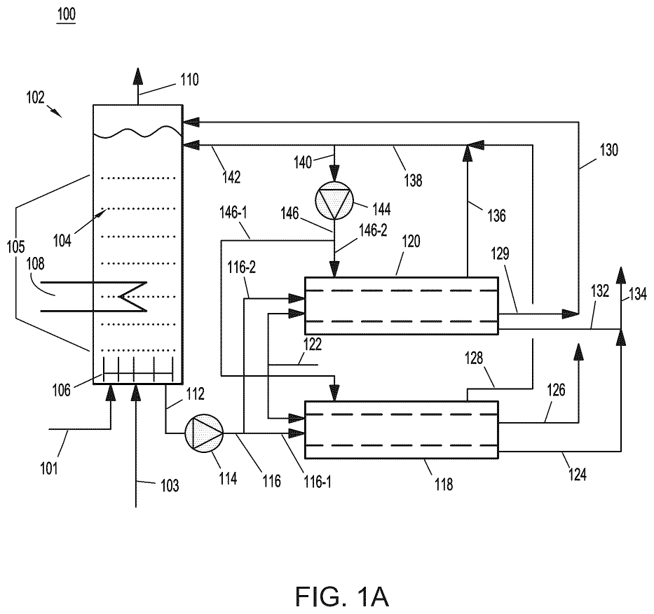

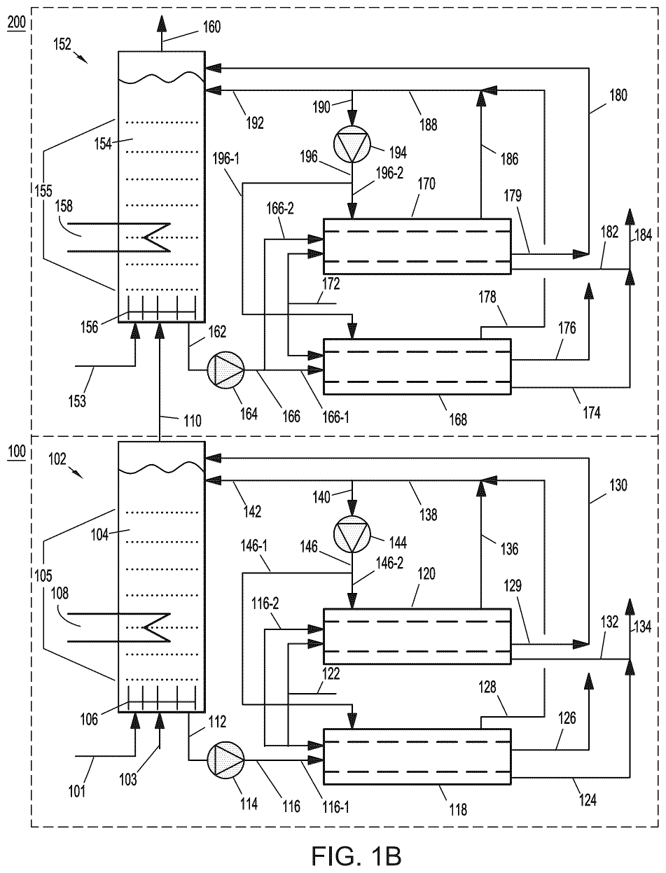

The non-limiting illustrative embodiments of the present disclosure will be specifically described below with reference to the accompanying drawings. For the purpose of clarity, some steps leading up to the production of the hydrogenation effluent comprising ethylene as illustrated in FIGS. 1A and 1B may be omitted. In other words, one or more well-known processing steps which are not illustrated but are well-known to those of ordinary skill in the art have not been included in the figures. This is not intended to be interpreted as a limitation of any particular embodiment, or illustration, or scope of the claims.

Referring now to the drawings in more detail, FIGS. 1A and 1B illustrate details of systems and processes for improved production of a hydrogenation effluent comprising ethylene from acetylene utilizing a system including at least one or more acetylene absorption and hydrogenation slurry reactors, one or more liquid-solid separation units, and a plurality of pumps. It is to be understood that the system including at least the one or more acetylene absorption and hydrogenation slurry reactors, the one or more liquid-solid separation units, and the plurality of pumps is not limited to the configuration of the embodiments shown in FIGS. 1A and 1B, and other configurations are contemplated herein.

Referring now to FIG. 1A, a system 100 includes an acetylene absorption and hydrogenation slurry reactor 102 (hereinafter referred to as “slurry reactor 102”) for receiving an acetylene-rich gas stream 101 and a hydrogen stream 103 in a bottom end of slurry reactor 102 and a third regenerated slurry catalyst 130 and a fifth recycled solvent stream 142 as discussed below in a top end of slurry reactor 102 as part of a continuous solvent recycle and catalyst regeneration loop. However, these entry points are merely illustrative and any point of entry of acetylene-rich gas stream 101, hydrogen stream 103, third regenerated slurry catalyst 130 and fifth recycled solvent stream 142 into slurry reactor 102 is contemplated. In some embodiments, acetylene-rich gas stream 101 and hydrogen stream 103 are combined into one gas stream containing both acetylene and hydrogen.

In some embodiments, acetylene-rich gas stream 101 is obtained from the pyrolysis of a light hydrocarbon feed stream comprising methane such as, for example, natural gas. However, this is merely illustrative and any industrial process for producing an acetylene-rich gas stream is contemplated herein. In some embodiments, acetylene-rich gas stream 101 can contain at least a high concentration of acetylene as well as ethylene, methane, hydrogen and other light hydrocarbons. In some embodiments, acetylene-rich gas stream 101 can contain from about 0.1 wt. % to about 20 wt. % of acetylene.

Hydrogen stream 103 includes hydrogen, which is contained in a hydrogen “treat gas,” for injecting into slurry reactor 102 to allow sufficient hydrogen vapor pressure in slurry reactor 102 for at least the hydrogenation reaction as discussed below. The treat gas can be either pure hydrogen or a hydrogen-containing gas, which is a gas stream containing hydrogen in an amount that is sufficient for the intended reaction(s), optionally including one or more other gases (e.g., nitrogen and light hydrocarbons such as methane). The treat gas stream introduced into a reaction stage can contain at least about 50 vol. % or at least about 75 vol. % hydrogen. Optionally, the hydrogen treat gas can be substantially free (less than about 1 vol. %) of impurities such as H₂S and NH₃ and/or such impurities can be substantially removed from a treat gas prior to use. Hydrogen can be supplied co-currently with the input feed to slurry reactor 102 or separately via a separate gas conduit.

In some embodiments, slurry reactor 102 may be cylindrical in shape. In some embodiments, slurry reactor 102 may comprise a bottom end and a top end. In a non-limiting illustrative embodiment, the bottom end and the top end may be hemispherical or conical in shape. Suitable slurry reactors for slurry reactor 102 of system 100 include, for example, continuous stirred tank reactors, fluidized bed reactors, spouted bed reactors, spray reactors, slurry bubble column reactors, liquid recirculation reactors, slurry recirculation reactors, and combinations thereof. In some embodiments, slurry reactor 102 is an upright cylindrical separative reactor. One or more slurry reactors may be utilized in parallel or in series as discussed below with reference to FIG. 1B.

In some embodiments, slurry reactor 102 is a slurry bubble column reactor. In some embodiments, a slurry bubble column reactor is an upflow reactor in which acetylene-rich gas stream 101 and hydrogen stream 103 are introduced into slurry reactor 102 at or near the bottom of slurry reactor 102. Although it is shown that acetylene-rich gas stream 101 and hydrogen stream 103 are individually fed into slurry reactor 102, it is contemplated that any arrangement for introducing acetylene-rich gas stream 101 and hydrogen stream 103 into slurry reactor 102 can be utilized in the present disclosure.

In some embodiments, slurry reactor 102 of system 100 further includes a gas sparger 106 (also referred to as a flow distributor) for receiving acetylene-rich gas stream 101 and hydrogen stream 103. In some embodiments, gas sparger 106 may be of various configurations such as, for example, a ring-type sparger with multiple orifices, a sintered metal plate or sintered metal distributing pipe(s). In some embodiments, gas sparger 106 can include a plurality of gas spargers for distributing acetylene-rich gas stream 101 and hydrogen stream 103. The function of gas sparger 106 is to uniformly distribute acetylene-rich gas stream 101 and hydrogen stream 103 in slurry reactor 102. In some embodiments, gas sparger 106 is located at a bottom end of slurry reactor 102.

In some embodiments, slurry reactor 102 includes a slurry catalyst bed 105 (also referred to as “reaction zone”) for further receiving a slurry catalyst 104 comprising slurry catalyst particles and a liquid solvent generated from third regenerated slurry catalyst 130 and fifth recycled solvent stream 142. In some embodiments, slurry catalyst 104 contains a slurry of a suitable liquid solvent and solid catalyst particles, where the liquid solvent has a high selectivity to acetylene and low solubility for ethylene and the solid catalyst particles are suitable for an acetylene selective hydrogenation reaction.

The slurry hydroconversion process as discussed below uses slurry catalyst 104 which includes a liquid solvent and dispersed catalyst particles. In some embodiments, the catalyst can correspond to one or more catalytically active metals in particulate form and/or supported on particles. In some embodiments, the slurry hydroconversion catalyst may be a precursor thereof.

In some embodiments, the slurry hydroconversion catalyst is generally provided in the form of fine particulates or extrudates dispersed within the reactor liquid reaction medium and may be a supported catalyst, an unsupported catalyst, or a combination thereof. In some embodiments, the slurry hydroconversion catalyst comprises a metal selected from Group VIB, Group VIII of the Periodic Table, or a combination thereof. The slurry hydroconversion catalyst may be unsulfided or pre-sulfided before being added to the reactor. The slurry hydroconversion catalyst can comprise one or more different slurry hydroconversion catalysts as a single combined feed stream or as separate feeds to the reactor. In some embodiments, the slurry hydroconversion catalyst may have an average particle size of at least about 0.1 micron to about 5000 microns. In some embodiments, the slurry hydroconversion catalyst may have an average particle size of at least about 01 micron to about 2000 microns, e.g., from about 10 to about 1000 microns. In some embodiments, the slurry hydroconversion catalyst comprises a metal sulfide comprising one or more metals selected from the group consisting of molybdenum, nickel, cobalt and tungsten, and the slurry hydroconversion catalyst comprises particles having an average particle size of about 10 microns to about 1000 microns.

In some embodiments, the slurry hydroconversion catalyst is a slurry hydrogenation catalyst. Suitable slurry hydrogenation catalysts include, for example, an acetylene hydrogenation catalyst that is a catalyst selective for hydrogenating acetylene. The hydrogenation catalyst may be any known catalyst for selectively hydrogenating acetylene. Commercial catalysts for acetylene hydrogenation are widely available, and the present disclosure is not limited to any specific composition recited herein. In some embodiments, a hydrogenation catalyst can include a hydrogenation metal in an amount between about 0.01 wt. % to about 5.0 wt. % on a support, wherein the hydrogenation metal is selected from a Group VIII metal. In some embodiments, the metal can be platinum (Pt), palladium (Pd), nickel (Ni), or a mixture thereof. In some embodiments, a Group VIII metal is modified by one or more metals, selected from Group IB through IVA, such as zinc (Zn), indium (In), tin (Sn), lead (Pb), copper (Cu), silver (Ag), gold (Au) in an amount between about 0.01 and about 5 wt. %. Suitable supports include, for example, aluminum oxides (aluminas), pure or doped with other metal oxides, synthetic or natural (i.e., clays). In some embodiments, supports can be alpha-aluminas of various shapes and size (i.e., spheres, extrudates), with a high degree of conversion to the alpha phase.

In operation, acetylene-rich gas stream 101 and hydrogen stream 103 enter slurry reactor 102 and flow upwards via gas sparger 106 to slurry catalyst bed 105 and slurry catalyst 104 comprising slurry catalyst particles and a liquid solvent flow downward from third regenerated slurry catalyst 130 and fifth recycled solvent stream 142 to slurry catalyst bed 105. After allowing for sufficient mixing between slurry catalyst 104, acetylene-rich gas stream 101 and hydrogen stream 103, acetylene from acetylene-rich gas stream 101 and hydrogen from hydrogen stream 103 are extracted into the liquid solvent of slurry catalyst 104, which further react in slurry catalyst bed 105 to selectively produce an acetylene-lean gas effluent 110 and a spent slurry catalyst effluent 112 comprising spent catalyst particles and liquid solvent.

In some embodiments, acetylene-lean gas effluent 110 can be composed of about 0.001 wt. % to about 0.1 wt. % acetylene. Acetylene-lean gas effluent 110 exits through a top portion of slurry reactor 102 as an overhead stream that is rich in ethylene and having a reduced content of acetylene relative to acetylene-rich gas stream 101. Acetylene-lean gas effluent 110 may be passed to other processing and separation zones, the particulars of which are not necessary for an understanding and practicing of the present invention. For example, acetylene-lean gas effluent 110 may be sent to a separation unit external or internal to system 100 for extracting ethylene to send for further processing. Additionally, since acetylene-lean gas effluent 110 may include hydrogen, a portion of acetylene-lean gas effluent 110 may be recycled back to slurry reactor 102 to provide hydrogen for other hydrogenation reactions. In some embodiments, acetylene-lean gas effluent 110 may be passed to one or more other slurry reactors for further processing as discussed below with reference to FIG. 1B.

In some embodiments, acetylene-rich gas stream 101 and hydrogen stream 103 fed to slurry reactor 102 may undergo various hydroconversion reactions, including, for example, hydrocracking, hydrogenation, hydrodeoxygenation, hydrodesulfurization, hydrodenitrogenation, hydrodemetallization, hydrodechlorination, hydrodecarboxylation, hydrodecarbonylation, hydrodearomatization or a combination thereof. In one embodiment, the hydroconversion reaction is a hydrogenation reaction in which acetylene is selectively converted to ethylene.

The slurry hydroconversion process can be operated under slurry hydroconversion conditions such as slurry hydrogenation conditions. Suitable hydrogenation reaction conditions in slurry reactor 102 include, for example, a temperature that may range between about 20° C. and about 250° C., or between about 40° C. and about 200° C., or between about 40° C. and about 120° C. In addition, slurry reactor 102 can be operated at a high pressure which may range between approximately about 0.14 MPa (20 psig) and about 3.4 MPa (500 psig), or between about 1.0 MPa (150 psig) and about 2.8 MPa (400 psig). The liquid hour space velocity (LHSV) at the reactor inlet of slurry reactor 102 can range between about 1 and about 100 h⁻¹, or between about 5 and about 50 h⁻¹, or about 5 and about 25 h⁻¹.

In some embodiments, slurry reactor 102 can further include a heat exchanger 108 embedded in slurry reactor 102 to assist in regulating the desired temperature of the hydrogenation reaction. In some embodiments, heat exchanger 108 is configured to remove reaction heat generated during the portion of the acetylene from acetylene-rich gas stream 101 being converted to ethylene. For example, heat exchanger 108 can be configured to receive a heat transfer fluid provided at a temperature below the temperature of hydrogenation reaction in order to cool the reactor content to optimum temperatures for best acetylene conversion and ethylene selectivity during the reaction. In some embodiments, heat exchanger 108 may be a shell-and-tube, plate-fin, microchannel, spiral wound, or any other suitable heat exchanger. Although only one heat exchanger is shown in slurry reactor 102, any number of heat exchangers can be included in slurry reactor 102.

As discussed above, contacting acetylene-rich gas stream 101 and hydrogen stream 103 with slurry catalyst 104 comprising slurry catalyst particles and a liquid solvent in slurry catalyst bed 105 produces acetylene-lean gas effluent 110 and spent slurry catalyst effluent 112 comprising spent catalyst particles and liquid solvent. Spent slurry catalyst effluent 112 exits slurry reactor 102 at a bottom injection point and at an elevated temperature, e.g., a temperature ranging from about 100° C. to about 250° C., and is sent to a pump 114. Pump 114 can be any suitable pump for increasing the pressure of spent slurry catalyst effluent 112 for producing a pressurized spent slurry catalyst effluent 116. For example, pump 114 may be a rotary pump including an impeller, or alternatively may be any other suitable fluid pump, such as a centrifuge pump, a piston pump, and other positive displacement pump.

Pressurized spent slurry catalyst effluent 116 is split into two streams, namely, a first pressurized spent slurry catalyst effluent 116-1 and a second pressurized spent slurry catalyst effluent 116-2.

System 100 further includes a solvent recycle and a catalyst regeneration system including at least a first liquid-solid separation unit 118 and a second liquid-solid separation unit 120 to separate the liquid solvent from the spent catalyst particles in spent slurry catalyst effluent 112 exiting slurry reactor 102 and to recycle the liquid solvent and regenerate the spent catalyst particles thereby allowing a continuous solvent recycle and catalyst regeneration loop in system 100. In some embodiments, first liquid-solid separation unit 118 and second liquid-solid separation unit 120 can be back flushable filters for separating the liquid solvent from the spent catalyst particles. While two liquid-solid separation units are shown in system 100, any number of liquid-solid separation units are contemplated for use herein.

In non-limiting illustrative embodiments, system 100 can be operated in a manner by cycling between a normal filtration mode, i.e., a solvent recycle mode, and a catalyst regeneration mode. In some embodiments, first pressurized spent slurry catalyst effluent 116-1 is passed to first liquid-solid separation unit 118 where first liquid-solid separation unit 118 is operated in the normal filtration mode to separate the spent catalyst particles from the liquid solvent and produce a first recycled solvent stream 128 while retaining the spent catalyst particles inside first liquid-solid separation unit 118. First recycled solvent stream 128 will contain relatively little to no spent catalyst particles.

In some embodiments, second pressurized spent slurry catalyst effluent 116-2 is passed to second liquid-solid separation unit 120 where second liquid-solid separation unit 120 is operated in the normal filtration mode to separate the spent catalyst particles from the liquid solvent and produce a second recycled solvent stream 136 while retaining the spent catalyst particles inside second liquid-solid separation unit 120. Second recycled solvent stream 136 will contain relatively little to no spent catalyst particles.

First recycled solvent stream 128 can be combined with second recycled solvent stream 136 to form a third recycled solvent stream 138. Third recycled solvent stream 138 can then be split as a fourth recycled solvent stream 140 and fifth recycled solvent stream 142. Fifth recycled solvent stream 142 can continuously be introduced into slurry reactor 102.

In some embodiments, fourth recycled solvent stream 140 can be sent to a pump 144. Pump 144 can be any suitable pump for increasing the pressure of fourth recycled solvent stream 140 for producing a pressurized recycled solvent stream 146. For example, pump 144 may be a rotary pump including an impeller, or alternatively may be any other suitable fluid pump such as a centrifuge pump, a piston pump and other positive displacement pumps.

Pressurized recycled solvent stream 146 is split into two streams, namely, a first pressurized recycled solvent stream 146-1 and a second pressurized recycled solvent stream 146-2.

Next, depending on the pressure drop across first liquid-solid separation unit 118 and second liquid-solid separation unit 120, first liquid-solid separation unit 118 and second liquid-solid separation unit 120 can switch to operate in a catalyst regenerating mode. For example, when the pressure drop across either of first liquid-solid separation unit 118 or second liquid-solid separation unit 120 is at or below about 10 psi to about 25 psi, first liquid-solid separation unit 118 and second liquid-solid separation unit 120 can operate in the catalyst regenerating mode. Alternatively, when the pressure drop across one of first liquid-solid separation unit 118 and second liquid-solid separation unit 120 is above about 10 psi to about 25 psi then incoming pressurized spent slurry catalyst effluent 116 will be diverted to the particular liquid-solid separation unit which has the pressure drop below about psi 10 to about 25 psi. For example, in the case where the pressure drop across first liquid-solid separation unit 118 is above about 10 psi to about 25 psi, then incoming pressurized spent slurry catalyst effluent 116 will be diverted to second liquid-solid separation unit 120 as second pressurized spent slurry catalyst effluent 116-2 for solvent recycle and catalyst regeneration.

In some embodiments, when operating in the catalyst regenerating mode, first liquid-solid separation unit 118 or second liquid-solid separation unit 120 can include a catalyst regeneration gas inlet adapted to receive an oxidizing stream 122. In some embodiments, oxidizing stream 122 can enter first liquid-solid separation unit 118 or second liquid-solid separation unit 120 through a heating unit (not shown) configured to heat oxidizing stream 122 to a temperature sufficient to combust the spent catalyst particles to produce regenerated catalyst particles. Oxidizing stream 122 can contain, for example, air, oxygen, nitrogen, methane or combinations thereof, a hot steam stream or a steam/air mixture.

In some embodiments, the heating unit can be an air heater, which may include a resistive or inductive heating element configured to heat oxidizing stream 122 to generate a heated oxidizing stream. In some embodiments, the heating unit can be a steam heater, and may include a heating element, such as a resistive or inductive heating element configured to heat oxidizing stream 122 to generate a heated oxidizing stream. In some embodiments, the heating unit may include a heat exchanger configured to heat the steam using heat extracted from a high-temperature fluid, such as a fluid heated to about 500° C. or more. This fluid may be provided from a solar concentrator farm or a power plant.

In some embodiments, oxidizing stream 122 can be introduced into one or more of first liquid-solid separation unit 118 and second liquid-solid separation unit 120 to regenerate the spent catalyst particles captured during the course of operating first liquid-solid separation unit 118 and/or second liquid-solid separation unit 120 in the normal filtration mode. For example, during the slurry hydroconversion process discussed above, slurry catalyst 104 comprising slurry catalyst particles and a liquid solvent will become deactivated due to the presence of impurities such as heavy hydrocarbons such as C₄ and C₅₊ acetylenic and diolefinic species as well as coke and green oil formation thereby producing the spent catalyst. The spent catalyst can then be regenerated in first liquid-solid separation unit 118 and/or second liquid-solid separation unit 120 by exposing the spent catalyst particles to oxidizing stream 122 at appropriate high temperature and time duration conditions to burn and remove substantially all these impurities from the catalyst and provide a regenerated catalyst. In an illustrative embodiment, a temperature can range from about 500° C. to about 800° C., and a time period can range from about 1 hour to about 12 hours or even longer.

Accordingly, regenerating the spent catalyst particles generally comprises combustion of the spent catalyst particles in an oxidizing atmosphere to burn the impurities such as heavy hydrocarbons and coke deposits and redisperse active metal on the catalyst particles. Burning the impurities is an exothermic process that can supply the heat needed for the hydroconversion process to be carried out in slurry reactor 102, if necessary.

In some embodiments, the coke burn causes the spent catalyst particles to be heated to an elevated temperature, e.g., a temperature of from about 500° C. to about 800° C., to provide a heated regenerated catalyst particles relatively free or free of coke wherein the catalyst particles are heated, as well as a first heated gas effluent 124 from first liquid-solid separation unit 118 and a second heated gas effluent 132 from second liquid-solid separation unit 120. In some embodiments, first heated gas effluent 124 and second heated gas effluent 132 can be regenerator flue gas composed of, for example, carbon dioxide and nitrogen, which exit respective first liquid-solid separation unit 118 and second liquid-solid separation unit 120. In some embodiments, first heated gas effluent 124 and second heated gas effluent 132 can be combined as a third heated gas effluent 134 which can exit system 100 by way of, for example, an exhaust or vent.

In addition, during operation of the catalyst regeneration mode in one or more of first liquid-solid separation unit 118 and second liquid-solid separation unit 120, first pressurized recycled solvent stream 146-1 and second recycled solvent stream 146-2 will continuously flow back through respective first liquid-solid separation unit 118 and second liquid-solid separation unit 120 to re-slurry the regenerated catalyst particles and produce a first slurry catalyst 126 from first liquid-solid separation unit 118 and a second slurry catalyst 129 from second liquid-solid separation unit 120. In some embodiments, first slurry catalyst 126 and second slurry catalyst 129 can be combined as third regenerated slurry catalyst 130 and continuously introduced into slurry reactor 102 for use in the slurry hydroconversion process discussed above.

In a non-limiting illustrative embodiment, two or more slurry reactors can be used in series to significantly increase the overall conversion of acetylene to ethylene in producing a product stream with a relatively low concentration of acetylene. Accordingly, as illustrated in FIG. 1B, system 100 discussed above is operated in series with a system 200 to increase the overall conversion of acetylene to ethylene.

System 200 includes an acetylene absorption and hydrogenation slurry reactor 152 (hereinafter referred to as “slurry reactor 152”) for receiving acetylene-lean gas effluent 110 and a hydrogen stream 153 in a bottom end of slurry reactor 152 and a third regenerated slurry catalyst 180 and a fifth recycled solvent stream 192 in a top end of slurry reactor 152 as part of a continuous solvent recycle and catalyst regeneration loop. However, these entry points are merely illustrative and any point of entry of acetylene-lean gas effluent 110, hydrogen stream 153, third regenerated slurry catalyst 180 and fifth recycled solvent stream 192 into slurry reactor 152 is contemplated.

Hydrogen stream 153 includes hydrogen, which is contained in a hydrogen “treat gas,” for injecting into slurry reactor 152 to allow sufficient hydrogen vapor pressure in slurry reactor 152 for at least a hydrogenation reaction as discussed below. Hydrogen stream 153 can be similar to hydrogen stream 103 discussed above.

In some embodiments, slurry reactor 152 can be of a similar configuration and of a similar slurry reactor as discussed above for slurry reactor 102. In some embodiments, slurry reactor 152 is a slurry bubble column reactor.

In some embodiments, slurry reactor 152 of system 200 further includes a gas sparger 156 (also referred to as a flow distributor) for receiving acetylene-lean gas effluent 110 and hydrogen stream 153. In some embodiments, gas sparger 156 can be of a similar configuration and of a similar gas sparger as gas sparger 106 discussed above. The function of gas sparger 156 is to uniformly distribute acetylene-lean gas effluent 110 and hydrogen stream 153 in slurry reactor 152. In some embodiments, gas sparger 156 is located at a bottom end of slurry reactor 152.

In some embodiments, slurry reactor 152 includes a slurry catalyst bed 155 (also referred to as “reaction zone”) for further receiving slurry catalyst 154 comprising slurry catalyst particles and a liquid solvent generated from third regenerated slurry catalyst 180 and fifth recycled solvent stream 192. In some embodiments, slurry catalyst 154 contains a slurry of a suitable liquid solvent and solid catalyst particles, where the liquid solvent has a high selectivity to acetylene and low solubility for ethylene and the solid catalyst particles are suitable for an acetylene selective hydrogenation reaction.

The slurry hydroconversion process as discussed below uses slurry catalyst 154 which includes a liquid solvent composed of dispersed catalyst particles. In some embodiments, the catalyst can correspond to one or more catalytically active metals in particulate form and/or supported on particles. Slurry catalyst 154 can be the same as slurry catalyst 104 discussed above.

In operation, acetylene-lean gas effluent 110 and hydrogen stream 153 enter slurry reactor 152 and flow upwards via gas sparger 156 to slurry catalyst bed 155 and slurry catalyst 154 comprising slurry catalyst particles and a liquid solvent flow downward from third regenerated slurry catalyst 180 and fifth recycled solvent stream 192 to slurry catalyst bed 155. After allowing for sufficient mixing between slurry catalyst 154, acetylene-lean gas effluent 110 and hydrogen stream 153, acetylene from acetylene-lean gas effluent 110 and hydrogen from hydrogen stream 153 are extracted into the liquid solvent of slurry catalyst 154, which further react in slurry catalyst bed 155 to selectively produce a second acetylene-lean gas effluent 160 and a spent slurry catalyst effluent 162 comprising spent catalyst particles and liquid solvent. Suitable slurry hydroconversion conditions and slurry hydrogenation conditions can be similar to those discussed above.

In some embodiments, slurry reactor 152 can further include a heat exchanger 158 embedded in slurry reactor 152 to assist in regulating the desired temperature of the hydrogenation reaction. In some embodiments, heat exchanger 158 is configured to remove reaction heat generated during the portion of the acetylene from acetylene-lean gas effluent 110 being converted to ethylene. For example, heat exchanger 158 can be configured to receive a heat transfer fluid provided at a temperature below the temperature of hydrogenation reaction in order to cool the reactor content to an optimum temperature for best acetylene conversion and ethylene selectivity during the reaction. In some embodiments, heat exchanger 158 may be a shell-and-tube, plate-fin, microchannel, spiral wound, or any other suitable heat exchanger. Although only one heat exchanger is shown in slurry reactor 152, any number of heat exchangers can be included in slurry reactor 152.

Second acetylene-lean gas effluent 160 is a vapor stream that has a reduced content of acetylene and an increased content of ethylene relative to acetylene-lean gas effluent 110. Second acetylene-lean gas effluent 160 further includes hydrogen gas. In some embodiments, second acetylene-lean gas effluent 160 can be composed of greater than about 0.1 wt. % ethylene, or greater than about 0.5 wt. % ethylene or greater than about 1 wt. % ethylene, and hydrogen gas. In some embodiments, second acetylene-lean gas effluent 160 can be composed of about 0.1 wt. % to about 0.5 wt. % acetylene. Second acetylene-lean gas effluent 160 exits through a top portion of slurry reactor 152 as an overhead stream that is rich in ethylene and may be passed to other processing and separation zones, the particulars of which are not necessary for an understanding and practicing of the present invention. For example, second acetylene-lean gas effluent 160 may be sent to a separation unit for extracting ethylene to send for further processing. Additionally, since second acetylene-lean gas effluent 160 may include hydrogen, a portion of second acetylene-lean gas effluent 160 may be recycled back to slurry reactor 152 to provide hydrogen for other hydrogenation reactions.

As discussed above, contacting acetylene-lean gas effluent 110 and hydrogen stream 153 with slurry catalyst 154 comprising slurry catalyst particles and a liquid solvent in slurry catalyst bed 155 produces second acetylene-lean gas effluent 160 and spent slurry catalyst effluent 162 comprising spent catalyst particles and liquid solvent. Spent slurry catalyst effluent 162 exits slurry reactor 152 at a bottom injection point and at an elevated temperature, e.g., a temperature ranging from about 50° C. to about 250° C., and is sent to a pump 164. Pump 164 can be any suitable pump for increasing the pressure of spent slurry catalyst effluent 162 for producing a pressurized spent slurry catalyst effluent 166. For example, pump 164 may be a rotary pump including an impeller, or alternatively may be any other suitable fluid pump such as a centrifuge pump, a piston pump and other positive displacement pumps.

Pressurized spent slurry catalyst effluent 166 is split into two streams, namely, a first pressurized spent slurry catalyst effluent 166-1 and a second pressurized spent slurry catalyst effluent 166-2.

System 200 further includes a solvent recycle and a catalyst regeneration system including at least a first liquid-solid separation unit 168 and a second liquid-solid separation unit 170 to separate the liquid solvent from the spent catalyst particles in spent slurry catalyst effluent 162 exiting slurry reactor 152 and to recycle the liquid solvent and regenerate the spent catalyst particles thereby allowing a continuous solvent recycle and catalyst regeneration loop in system 200. In some embodiments, first liquid-solid separation unit 168 and second liquid-solid separation unit 170 can be back flushable filters. While two liquid-solid separation units are shown in system 200, any number of liquid-solid separation units are contemplated for use herein.

In non-limiting illustrative embodiments, system 200 can be operated in a manner by cycling between a normal filtration mode, i.e., a solvent recycle mode, and a catalyst regeneration mode. In some embodiments, first pressurized spent slurry catalyst effluent 166-1 is passed to first liquid-solid separation unit 168 where first liquid-solid separation unit 168 is operated in the normal filtration mode to separate the spent catalyst particles from the liquid solvent and produce a first recycled solvent stream 178 while retaining the spent catalyst particles inside first liquid-solid separation unit 168. First recycled solvent stream 178 will contain relatively little to no spent catalyst particles.

In some embodiments, second pressurized spent slurry catalyst effluent 166-2 is passed to second liquid-solid separation unit 170 where second liquid-solid separation unit 170 is operated in the normal filtration mode to separate the spent catalyst particles from the liquid solvent and produce a second recycled solvent stream 186 while retaining the spent catalyst particles inside second liquid-solid separation unit 170. Second recycled solvent stream 186 will contain relatively little to no spent catalyst particles.

First recycled solvent stream 178 can be combined with second recycled solvent stream 186 to form a third recycled solvent stream 188. Third recycled solvent stream 188 can then be split as a fourth recycled solvent stream 190 and fifth recycled solvent stream 192. Fifth recycled solvent stream 192 can continuously be introduced into slurry reactor 152.

In some embodiments, fourth recycled solvent stream 190 can be sent to a pump 194. Pump 194 can be any suitable pump for increasing the pressure of fourth recycled solvent stream 190 for producing a pressurized recycled solvent stream 196. For example, pump 194 may be a rotary pump including an impeller, or alternatively may be any other suitable fluid pump such as a centrifuge pump, a piston pump and other positive displacement pumps.

Pressurized recycled solvent stream 196 is split into two streams, namely, a first pressurized recycled solvent stream 196-1 and a second pressurized recycled solvent stream 196-2.

Next, depending on the pressure drop across first liquid-solid separation unit 168 and second liquid-solid separation unit 170, first liquid-solid separation unit 168 and second liquid-solid separation unit 170 can switch to operate in the catalyst regenerating mode. For example, when the pressure drop across either of first liquid-solid separation unit 168 or second liquid-solid separation unit 170 is at or below about 10 psi to about 25 psi, first liquid-solid separation unit 168 and second liquid-solid separation unit 170 can operate in the catalyst regenerating mode. Alternatively, when the pressure drop across one of first liquid-solid separation unit 168 and second liquid-solid separation unit 170 is above about 10 psi to about 25 psi then incoming pressurized spent slurry catalyst effluent 166 will be diverted to the particular liquid-solid separation unit which has the pressure drop below about 10 psi to about 25 psi. For example, in the case where the pressure drop across first liquid-solid separation unit 118 is above about 10 psi to about 25 psi, then incoming pressurized spent slurry catalyst effluent 166 will be diverted to second liquid-solid separation unit 170 as second pressurized spent slurry catalyst effluent 166-2 for solvent recycle and catalyst regeneration.

In some embodiments, when operating in the catalyst regenerating mode, first liquid-solid separation unit 168 or second liquid-solid separation unit 170 can include a catalyst regeneration gas inlet adapted to receive an oxidizing stream 172. In some embodiments, oxidizing stream 172 can enter first liquid-solid separation unit 168 or second liquid-solid separation unit 170 through a heating unit (not shown) configured to heat oxidizing stream 172 to a temperature sufficient to combust the spent catalyst particles to produce regenerated catalyst particles. Oxidizing stream 172 can contain, for example, air, oxygen, nitrogen, methane or combinations thereof, a hot steam stream or a steam/air mixture. In some embodiments, the heating unit can be a similar heating unit as discussed above.

In some embodiments, oxidizing stream 172 can be introduced into one or more of first liquid-solid separation unit 168 and second liquid-solid separation unit 170 to regenerate the spent catalyst particles captured during the course of operating first liquid-solid separation unit 168 and/or second liquid-solid separation unit 170 in the normal filtration mode. For example, during the slurry hydroconversion process discussed above, slurry catalyst 154 comprising slurry catalyst particles and a liquid solvent will become deactivated due to the presence of impurities such as heavy hydrocarbons such as C₄ and C₅₊ acetylenic and diolefinic species as well as coke and green oil formation thereby producing the spent catalyst. The spent catalyst can then be regenerated in first liquid-solid separation unit 168 and/or second liquid-solid separation unit 170 by exposing the spent catalyst particles to oxidizing stream 172 at appropriate high temperature and time duration conditions to burn and remove substantially all these impurities from the catalyst and provide a regenerated catalyst. In an illustrative embodiment, a temperature can range from about 500° C. to about 800° C., and a time period can range from about 1 hour to about 12 hours or even longer.

Accordingly, regenerating the spent catalyst particles generally comprises combustion of the spent catalyst particles in an oxidizing atmosphere to burn the impurities such as heavy hydrocarbons and coke deposits and redisperse active metal on the catalyst particles. Burning the impurities is an exothermic process that can supply the heat needed for the hydroconversion process to be carried out in slurry reactor 152, if necessary.

In some embodiments, the coke burn causes the spent catalyst particles to be heated to an elevated temperature, e.g., a temperature of from about 500° C. to about 800° C., to provide a heated regenerated catalyst particles relatively free or free of coke wherein the catalyst particles are heated, as well as a first heated gas effluent 174 from first liquid-solid separation unit 168 and a second heated gas effluent 182 from second liquid-solid separation unit 170. In some embodiments, first heated gas effluent 174 and second heated gas effluent 182 can be regenerator flue gas composed of, for example, carbon dioxide and nitrogen, which exit respective first liquid-solid separation unit 168 and second liquid-solid separation unit 170. In some embodiments, first heated gas effluent 174 and second heated gas effluent 182 can be combined as third heated gas effluent 184 which can exit system 200 by way of, for example, an exhaust or vent.

In addition, during operation of the catalyst regeneration mode in one or more of first liquid-solid separation unit 168 and second liquid-solid separation unit 170, first pressurized recycled solvent stream 196-1 and second recycled solvent stream 196-2 will continuously flow backwards through respective first liquid-solid separation unit 168 and second liquid-solid separation unit 170 to re-slurry the regenerated catalyst particles and produce a first regenerated slurry catalyst 176 from first liquid-solid separation unit 168 and a second regenerated slurry catalyst 179 from second liquid-solid separation unit 170. In some embodiments, first regenerated slurry catalyst 176 and second regenerated slurry catalyst 179 can be combined as third regenerated slurry catalyst 180 and continuously introduced into slurry reactor 152 for use in the slurry hydroconversion process discussed above.

According to an aspect of the disclosure, a system, comprises:
a first slurry reactor configured to receive an acetylene-rich gas stream and a first hydrogen stream as one stream or separate streams flowing upwards to a reaction zone and a first slurry catalyst comprising catalyst particles and a liquid solvent flowing downwards to the reaction zone,
wherein the liquid solvent extracts acetylene in the acetylene-rich gas stream and hydrogen in the first hydrogen stream and at least a portion of the acetylene is converted to ethylene in the presence of the first slurry catalyst and the hydrogen under hydrogenation reaction conditions to generate an acetylene-lean gas effluent and a first spent slurry catalyst comprising spent catalyst particles and the liquid solvent.

In one or more additional non-limiting illustrative embodiments, as may be combined with one or more of the preceding paragraphs, the system further comprises one or more gas spargers located at a bottom end of the first slurry reactor for uniformly distributing the acetylene-rich gas stream and the first hydrogen stream in the first slurry reactor, wherein the first slurry reactor is a slurry bubble column reactor.

In one or more additional non-limiting illustrative embodiments, as may be combined with one or more of the preceding paragraphs, the system further comprises one or more heat exchangers located in the first slurry reactor each configured to remove reaction heat generated when the portion of the acetylene is converted to ethylene.

In one or more additional non-limiting illustrative embodiments, as may be combined with one or more of the preceding paragraphs, the system further comprises one or more liquid-solid separation and catalyst regeneration units, in fluid communication with the first slurry reactor, each configured to produce a recycled liquid solvent stream and a regenerated slurry catalyst stream comprising regenerated catalyst particles and a recycled liquid solvent based, in part, on the first spent slurry catalyst.

In one or more additional non-limiting illustrative embodiments, as may be combined with one or more of the preceding paragraphs, the one or more liquid-solid separation and catalyst regeneration units comprise one or more back flushable filters.

In one or more additional non-limiting illustrative embodiments, as may be combined with one or more of the preceding paragraphs, the one or more back flushable filters comprise a first back flushable filter and a second back flushable filter.

In one or more additional non-limiting illustrative embodiments, as may be combined with one or more of the preceding paragraphs, the system further comprises:

a second slurry reactor configured to receive the acetylene-lean gas effluent from the first slurry reactor and a second hydrogen stream flowing upwards to a reaction zone and a second slurry catalyst comprising catalyst particles and a liquid solvent flowing downwards to the reaction zone, wherein the liquid solvent extracts acetylene in the acetylene-lean gas effluent and hydrogen in the second hydrogen stream and at least a portion of the acetylene is converted to ethylene in the presence of the second slurry catalyst and the hydrogen under hydrogenation reaction conditions to generate an acetylene-lean gas effluent and a second spent slurry catalyst comprising spent catalyst particles and the liquid solvent.

In one or more additional non-limiting illustrative embodiments, as may be combined with one or more of the preceding paragraphs, the system further comprises one or more liquid-solid separation and catalyst regeneration units, in fluid communication with the second slurry reactor, each configured to produce a recycled liquid solvent stream and a regenerated slurry catalyst stream comprising regenerated catalyst particles and a recycled liquid solvent based, in part, on the second spent slurry catalyst.

In one or more additional non-limiting illustrative embodiments, as may be combined with one or more of the preceding paragraphs, the one or more liquid-solid separation and catalyst regeneration units comprise one or more back flushable filters.

According to another aspect of the disclosure, a continuous process comprises:
passing an acetylene-rich gas stream and a hydrogen stream as one stream or as separate streams to a slurry reactor flowing upwards to a reaction zone in the slurry reactor,
passing a slurry catalyst comprising catalyst particles and a liquid solvent to the slurry reactor flowing downwards to the reaction zone in the slurry reactor, and
processing acetylene in the acetylene-rich gas stream and hydrogen in the hydrogen stream to convert at least a portion of the acetylene to ethylene in the presence of the slurry catalyst and the hydrogen under hydrogenation reaction conditions to generate an acetylene-lean gas effluent and a spent slurry catalyst comprising spent catalyst particles and the liquid solvent.

In one or more additional non-limiting illustrative embodiments, as may be combined with one or more of the preceding paragraphs, the slurry reactor is a slurry bubble column and the process further comprises flowing the acetylene-rich gas stream and the hydrogen stream upwards using one or more gas spargers.

In one or more additional non-limiting illustrative embodiments, as may be combined with one or more of the preceding paragraphs, processing the acetylene and the hydrogen comprises extracting the acetylene and the hydrogen by the liquid solvent and reacting the acetylene with the hydrogen in the presence of the slurry catalyst under the hydrogenation reaction conditions to convert at least a portion of the acetylene to ethylene and generate the acetylene-lean gas effluent and the spent slurry catalyst comprising spent catalyst particles and the liquid solvent.

In one or more additional non-limiting illustrative embodiments, as may be combined with one or more of the preceding paragraphs, the continuous process further comprises passing the spent slurry catalyst comprising spent catalyst particles and the liquid solvent to one or more liquid-solid separation and catalyst regeneration units to produce a recycled liquid solvent stream and a regenerated slurry catalyst stream comprising regenerated catalyst particles and a recycled liquid solvent stream.

In one or more additional non-limiting illustrative embodiments, as may be combined with one or more of the preceding paragraphs, passing the spent slurry catalyst comprising spent catalyst particles and the liquid solvent to one or more liquid-solid separation and catalyst regeneration units comprises splitting the spent slurry catalyst to a first spent slurry catalyst and a second spent slurry catalyst and passing the first spent slurry catalyst to a first back flushable filter and passing the second spent slurry catalyst to a second back flushable filter.

In one or more additional non-limiting illustrative embodiments, as may be combined with one or more of the preceding paragraphs, passing the first spent slurry catalyst to the first back flushable filter further comprises filtering the spent catalyst particles from the first spent slurry catalyst to produce first filtered spent catalyst particles and a first recycled liquid solvent stream, and passing the second spent slurry catalyst to the second back flushable filter further comprises filtering the spent catalyst particles from the second spent slurry catalyst to produce second filtered spent catalyst particles and a second recycled liquid solvent stream.

In one or more additional non-limiting illustrative embodiments, as may be combined with one or more of the preceding paragraphs, passing the first spent slurry catalyst to the first back flushable filter is responsive to a pressure drop across the second back flushable filter being greater than 10 psi to about 25 psi.

In one or more additional non-limiting illustrative embodiments, as may be combined with one or more of the preceding paragraphs, the continuous process further comprises combusting the first filtered spent catalyst particles in the first back flushable filter to produce first regenerated catalyst particles and combusting the second filtered spent catalyst particles in the second back flushable filter to produce second regenerated catalyst particles.

In one or more additional non-limiting illustrative embodiments, as may be combined with one or more of the preceding paragraphs, the continuous process further comprises combining the first recycled liquid solvent stream and the second recycled liquid solvent stream to form a third recycled liquid solvent stream and passing a first portion of the third recycled liquid solvent stream to the first back flushable filter to produce a first regenerated catalyst slurry stream and passing a second portion of the third recycled liquid solvent stream to the second back flushable filter to produce a second regenerated catalyst slurry stream.

In one or more additional non-limiting illustrative embodiments, as may be combined with one or more of the preceding paragraphs, the recycled liquid solvent stream and the regenerated slurry catalyst stream are part of a continuous recycled solvent and catalyst regeneration loop.

In one or more additional non-limiting illustrative embodiments, as may be combined with one or more of the preceding paragraphs, the continuous process further comprises:
passing the acetylene-lean gas effluent and another hydrogen stream to another slurry reactor flowing upwards to another reaction zone in the other slurry reactor,
passing another slurry catalyst comprising catalyst particles and a liquid solvent to the other slurry reactor flowing downwards to the reaction zone in the other slurry reactor,
processing acetylene in the acetylene-lean gas effluent and hydrogen in the other hydrogen stream to convert at least a portion of the acetylene to ethylene in the presence of the other slurry catalyst and the hydrogen under hydrogenation reaction conditions to generate another acetylene-lean gas effluent having a reduced content of acetylene and an increased content of ethylene relative to the acetylene-lean gas effluent and another spent slurry catalyst comprising spent catalyst particles and the liquid solvent, and
passing the other spent slurry catalyst comprising spent catalyst particles and the liquid solvent to one or more other liquid-solid separation units to produce another recycled liquid solvent stream and another regenerated slurry catalyst stream comprising regenerated catalyst particles and a recycled liquid solvent.

Various features disclosed herein are, for brevity, described in the context of a single embodiment, but may also be provided separately or in any suitable sub-combination. All combinations of the embodiments are specifically embraced by the illustrative embodiments disclosed herein just as if each and every combination was individually and explicitly disclosed. In addition, all sub-combinations listed in the embodiments describing such variables are also specifically embraced by the present disclosure and are disclosed herein just as if each and every such sub-combination was individually and explicitly disclosed herein.

While the above description contains many specifics, these specifics should not be construed as limitations of the invention, but merely as exemplifications of preferred embodiments thereof. Those skilled in the art will envision many other embodiments within the scope and spirit of the invention as defined by the claims appended hereto.

What is claimed is:

1. A system, comprising:
a first slurry reactor configured to receive an acetylene-rich gas stream and a first hydrogen stream as one stream or separate streams flowing upwards to a reaction zone and a first slurry catalyst comprising catalyst particles and a liquid solvent flowing downwards to the reaction zone, wherein the liquid solvent extracts acetylene in the acetylene-rich gas stream and hydrogen in the first hydrogen stream and at least a portion of the acetylene is converted to ethylene in the presence of the first slurry catalyst and the hydrogen under hydrogenation reaction conditions to generate an acetylene-lean gas effluent and a first spent slurry catalyst comprising spent catalyst particles and the liquid solvent.

2. The system according to claim 1, further comprising one or more gas spargers located at a bottom end of the first slurry reactor for uniformly distributing the acetylene-rich gas stream and the first hydrogen stream in the first slurry reactor, wherein the first slurry reactor is a slurry bubble column reactor.

3. The system according to claim 1, further comprising one or more heat exchangers located in the first slurry reactor each configured to remove reaction heat generated when the portion of the acetylene is converted to ethylene.

4. The system according to claim 1, further comprising one or more liquid-solid separation and catalyst regeneration units, in fluid communication with the first slurry reactor, each configured to produce a recycled liquid solvent stream and a regenerated slurry catalyst stream comprising regenerated catalyst particles and a recycled liquid solvent based, in part, on the first spent slurry catalyst.

5. The system according to claim 4, wherein the one or more liquid-solid separation and catalyst regeneration units comprise one or more back flushable filters.

6. The system according to claim 5, wherein the one or more back flushable filters comprise a first back flushable filter and a second back flushable filter.

7. The system according to claim 1, further comprising:
a second slurry reactor configured to receive the acetylene-lean gas effluent from the first slurry reactor and a second hydrogen stream flowing upwards to a reaction zone and a second slurry catalyst comprising catalyst particles and a liquid solvent flowing downwards to the reaction zone, wherein the liquid solvent extracts acetylene in the acetylene-lean gas effluent and hydrogen in the second hydrogen stream and at least a portion of the acetylene is converted to ethylene in the presence of the second slurry catalyst and the hydrogen under hydrogenation reaction conditions to generate an acetylene-lean gas effluent and a second spent slurry catalyst comprising spent catalyst particles and the liquid solvent.

8. The system according to claim 7, further comprising one or more liquid-solid separation and catalyst regeneration units, in fluid communication with the second slurry reactor, each configured to produce a recycled liquid solvent stream and a regenerated slurry catalyst stream comprising regenerated catalyst particles and a recycled liquid solvent based, in part, on the second spent slurry catalyst.

9. The system according to claim 8, wherein the one or more liquid-solid separation and catalyst regeneration units comprise one or more back flushable filters.

10. A continuous process, comprising:
passing an acetylene-rich gas stream and a hydrogen stream as one stream or as separate streams to a slurry reactor flowing upwards to a reaction zone in the slurry reactor;
passing a slurry catalyst comprising catalyst particles and a liquid solvent to the slurry reactor flowing downwards to the reaction zone in the slurry reactor; and
processing acetylene in the acetylene-rich gas stream and hydrogen in the hydrogen stream to convert at least a portion of the acetylene to ethylene in the presence of the slurry catalyst and the hydrogen under hydrogenation reaction conditions to generate an acetylene-lean gas effluent and a spent slurry catalyst comprising spent catalyst particles and the liquid solvent.

11. The continuous process according to claim 10, wherein the slurry reactor is a slurry bubble column and the process further comprises flowing the acetylene-rich gas stream and the hydrogen stream upwards using one or more gas spargers.

12. The continuous process according to claim 10, wherein processing the acetylene and the hydrogen comprises extracting the acetylene and the hydrogen by the liquid solvent and reacting the acetylene with the hydrogen in the presence of the slurry catalyst under the hydrogenation reaction conditions to convert at least a portion of the acetylene to ethylene and generate the acetylene-lean gas effluent and the spent slurry catalyst comprising spent catalyst particles and the liquid solvent.

13. The continuous process according to claim 10, further comprising passing the spent slurry catalyst comprising spent catalyst particles and the liquid solvent to one or more liquid-solid separation and catalyst regeneration units to produce a recycled liquid solvent stream and a regenerated slurry catalyst stream comprising regenerated catalyst particles and a recycled liquid solvent stream.

14. The continuous process according to claim 13, wherein passing the spent slurry catalyst comprising spent catalyst particles and the liquid solvent to one or more liquid-solid separation and catalyst regeneration units comprises splitting the spent slurry catalyst to a first spent slurry catalyst and a second spent slurry catalyst and passing the first spent slurry catalyst to a first back flushable filter and passing the second spent slurry catalyst to a second back flushable filter.

15. The continuous process according to claim 14, wherein passing the first spent slurry catalyst to the first back flushable filter further comprises filtering the spent catalyst particles from the first spent slurry catalyst to produce first filtered spent catalyst particles and a first recycled liquid solvent stream, and passing the second spent slurry catalyst to the second back flushable filter further comprises filtering the spent catalyst particles from the second spent slurry catalyst to produce second filtered spent catalyst particles and a second recycled liquid solvent stream.

16. The continuous process according to claim 14, wherein the passing the first spent slurry catalyst to the first back flushable filter is responsive to a pressure drop across the second back flushable filter being greater than 10 psi to about 25 psi.

17. The continuous process according to claim 15, further comprising combusting the first filtered spent catalyst particles in the first back flushable filter to produce first regenerated catalyst particles and combusting the second filtered spent catalyst particles in the second back flushable filter to produce second regenerated catalyst particles.

18. The continuous process according to claim 15, further comprising combining the first recycled liquid solvent stream and the second recycled liquid solvent stream to form a third recycled liquid solvent stream and passing a first portion of the third recycled liquid solvent stream to the first back flushable filter to produce a first regenerated catalyst slurry stream and passing a second portion of the third recycled liquid solvent stream to the second back flushable filter to produce a second regenerated catalyst slurry stream.

19. The continuous process according to claim 13, wherein the recycled liquid solvent stream and the regenerated slurry catalyst stream are part of a continuous recycled solvent and catalyst regeneration loop.

20. The continuous process according to claim 10, further comprising:
passing the acetylene-lean gas effluent and another hydrogen stream to another slurry reactor flowing upwards to another reaction zone in the other slurry reactor;
passing another slurry catalyst comprising catalyst particles and a liquid solvent to the other slurry reactor flowing downwards to the reaction zone in the other slurry reactor;
processing acetylene in the acetylene-lean gas effluent and hydrogen in the other hydrogen stream to convert at least a portion of the acetylene to ethylene in the presence of the other slurry catalyst and the hydrogen under hydrogenation reaction conditions to generate another acetylene-lean gas effluent having a reduced content of acetylene and an increased content of ethylene relative to the acetylene-lean gas effluent and another spent slurry catalyst comprising spent catalyst particles and the liquid solvent; and
passing the other spent slurry catalyst comprising spent catalyst particles and the liquid solvent to one or more other liquid-solid separation units to produce another recycled liquid solvent stream and another regenerated slurry catalyst stream comprising regenerated catalyst particles and a recycled liquid solvent.
